(19) United States
(12) Patent Application Publication
(10) Pub. No.: US 2005/0182260 A1
(43) Pub. Date: Aug. 18, 2005

(54) Process for the preparation of cyclic imides in the presence of polyphosphoric acid

(76) Inventors: Werner Mederski, Zwingenberg (DE); Manfred Baumgarth, Darmstadt (DE); Martina Germann, Brombachtral (DE); Dieter Kux, Darmstadt (DE); Thomas Weitzel, Liederbach a. Ts. (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/514,888

(22) PCT Filed: Apr. 7, 2003
(86) PCT No.: PCT/EP03/03584

(30) Foreign Application Priority Data
May 18, 2002 (DE) 102 22 277.0

(51) Int. Cl. C07D 211/84; C07D 207/40

(52) U.S. Cl. 546/296; 548/545

(57) ABSTRACT

The present invention relates to a novel process for the preparation of N-substituted cyclic imides. N-substituted cyclic imides are valuable intermediates which can be employed, for example, for the synthesis of pharmacologically valuable compounds.

The present invention relates to a novel process for the preparation of N-substituted cyclic imides. N-substituted cyclic imides are valuable intermediates which can be employed, for example, for the synthesis of pharmacologically valuable compounds.

According to the literature, N-phenyl-substituted cyclic imides can be prepared in a 2-3-step process by reaction of anilines with the cyclic anhydrides of dicarboxylic acids. To this end, firstly, in a first step, the aniline is reacted with the cyclic dicarboxylic anhydride with cleavage of the anhydride ring to form the corresponding open-chain monoamide and is worked up. The monoamide obtained is subsequently, in a second step, reacted with carboxylic acid activators (via a mixed anhydride), such as N,N′-disuccinimidyl oxalate (Kometani T, Fitz T, Watt D S; Tet. Lett. 1986, 27, 919), acetic anhydride (Stiz D S, Souza M M, Golin V, Neto R A S, Correa R, Nunes R J, Yunes R A, Cechinel-Filho V; Pharmazie 2000, 55, 12; Wanner M J, Koomen G-J; Tetrahedron 1991, 47, 8431; Akula M R, Kabalka G W; Synth. Commun. 1998, 28, 2063; Shemchuk L A, Chernykh V P, Ivanova I L, Snitkovskii E L, Zhirov M V, Turov A V; Russ. J. Org. Chem. 1999, 35, 286) or thionyl chloride (Caulfield W L, Gibson S, Rae D R; J. Chem. Soc., Perkin Trans 1 1996, 545), to give the corresponding N-substituted cyclic imides.

JP 62212361 describes the preparation of cyclic imides by reaction of aniline and dicarboxylic anhydride in toluene at 50-160° C. in the presence of ion exchanger resins. Under these conditions, only ortho-diamines can be reacted in one step with glutaric anhydride to give 1-aminoarylpiperidine-2,6-diones.

Hoey G B et al. describe the reaction of aniline and o-methylaniline with glutaric or succinic acid under pressure, distillation of the resultant water or azeotropic removal of the water formed [J. Am. Chem. Soc. 1951, 4473]. With succinic acid, in no case was a cyclic imide obtained. With glutaric acid, cyclic imide was obtained, if this product was obtained at all, in a maximum amount of 20%.

As described, the known processes for the preparation of cyclic imides require at least 2 reaction steps to be carried out and/or result in reaction mixtures, which makes work-up of the products obtained in each case necessary. If a one-step reaction process is described, this results, if the cyclic imide is obtained at all, in product mixtures which have to be purified. In addition, cyclic imide is only obtained in low yields.

The object of the present invention was to provide an improved process for the synthesis of N-substituted cyclic imides which avoids the above-described disadvantages of the previous processes. In particular, the process should be simplified and the yield increased.

Surprisingly, it has been found that N-substituted cyclic imides can be obtained in a one-step process and in high yield if the primary amine is reacted directly with the corresponding ring-forming dicarboxylic acid in the presence of polyphosphoric acid. The present invention therefore relates to a process for the preparation of N-substituted cyclic imides which is characterised in that a primary amine is reacted with a dicarboxylic acid in the presence of polyphosphoric acid.

Polyphosphoric acid (PPA) is a mixture of up to 85% of phosphorus pentoxide and orthophosphoric acid and also linear polyphosphoric acid (Rowlands D A; Synth. Reagents 1985, 6, 156)

Suitable as primary amine are unbranched and branched alkylamines and arylamines, which may be unsubstituted and substituted. As arylamines, preference is given to unsubstituted and substituted aniline. Particular preference is given to substituted or unsubstituted aniline of the general formula I. 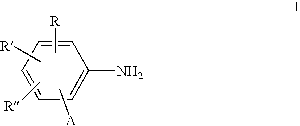
in which
R, R′, R″, independently of one another, are H, F, Cl, Br, I, alkyl, O-alkyl, —(C═O)alkyl, O—(C═O)alkyl, aryl, COOH, —(C═O)aryl, OCF₃, CF₃, CN, OCHF₂ or2,3-CH═CH—CH═CH—,
A is H, NO₂, NH₂ or NH—(C═O)—R¹,
alkyl is unbranched or branched alkyl having 1-6 C atoms,
aryl is phenyl or thienyl, each of which is unsubstituted or monosubstituted by alkyl, O-alkyl, CF₃,
R¹ is 2-phenoxy-2-aryl(or alkyl)acetamide or 2-phenylamino-2-aryl(or alkyl)acetamide

Alkyl is unbranched (linear) or branched, and has 1, 2, 3, 4, 5 or 6 C atoms. Alkyl preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3-or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, furthermore preferably, for example, trifluoromethyl.

Alkyl is very particularly preferably methyl, ethyl, propyl, isopropyl, butyl, isobutyl, sec-butyl, tert-butyl, pentyl, hexyl or trifluoromethyl.

A can be in the ortho-, meta- or para-position (4-position) to the primary amino group. A is preferably in the 4-position to the amino group. A is particularly preferably a nitro group and is in the 4-position to the primary amino group.

Suitable as dicarboxylic acid are unbranched and branched alkanes or alkenes which have an aliphatic chain containing 2, 3, 4 or 5 C atoms between the 2 carboxyl groups and are capable of forming a cyclic imide with the primary amine. Examples are saturated aliphatic dicarboxylic acids, such as succinic acid, glutaric acid, adipic acid, pimelic acid, but also dicarboxylic acids which contain one or more double bonds, such as, for example, maleic acid. Preference is given to dicarboxylic acids which have an aliphatic chain containing 2 or 3 C atoms between the 2 carboxyl groups, in particular maleic acid, succinic acid and substituted and unsubstituted glutaric acid. If branched glutaric acid is used, one or 2 of the H atoms in the 3-position is preferably substituted by alkyl having 1 to 6 C atoms or aryl.

In the reaction according to the invention of a primary amine of the formula I with one of the preferred dicarboxylic acids, the reaction product obtained is a cyclic imide of the general formula II 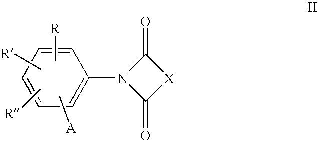
in which
R, R′, R″, independently of one another, are H, F, Cl, Br, I, alkyl, O-alkyl, —(C═O)alkyl, O—(C═O)alkyl, aryl, COOH, —(C═O)aryl, OCF₃, CF₃, CN, OCHF₂ or2,3-CH═CH—CH═CH—,
A is H, NO₂, NH₂ or NH—(C═O)—R¹,
x is —CH₂CH₂CH₂—, —CH₂CH₂—, —CH═CH—, —CH₂C(alkyl)₂CH₂—, —CH₂CH(alkyl)CH₂— or —CH₂CH-aryl-CH₂—,
alkyl is unbranched or branched alkyl having 1-6 C atoms,
aryl is phenyl or thienyl, each of which is unsubstituted or monosubstituted by alkyl, O-alkyl, CF₃,
R¹ is 2-phenoxy-2-aryl(or alkyl)acetamide or 2-phenylamino-2-aryl(or alkyl)acetamide.

In particular, the compounds of the formula II are valuable intermediates which can serve, for example, for the preparation of certain 2-phenoxy-2-aryl(or alkyl)acetamides or 2-phenylamino-2-aryl(or alkyl)acetamides, which act as inhibitors of coagulation Xa and VIIa. Compounds of this type are described, for example, in the pending German patent application No. 101 02322.

The reaction scheme whence scratch that the reaction sequence is depicted below for the particularly preferred glutaric acid (III), succinic acid (IV) and maleic acid (V) (reaction scheme 1).

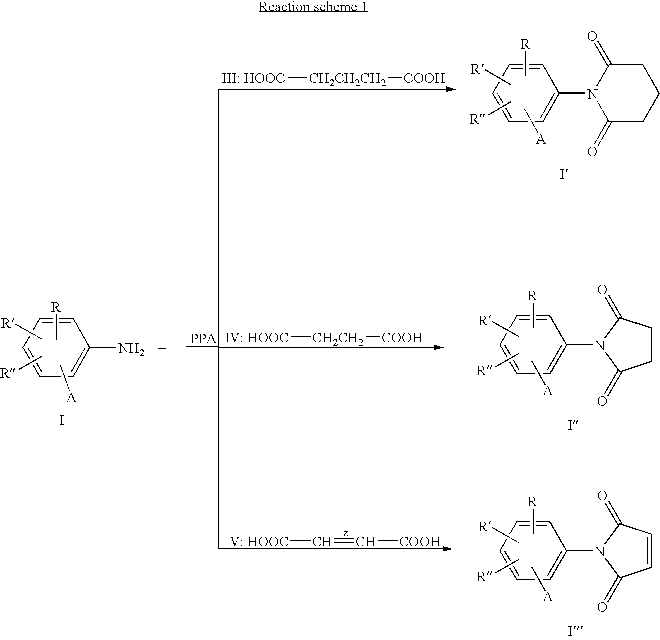

The process according to the invention can be carried out in a simple manner, preferably by bringing equimolar amounts of the two reactants to reaction with stirring in PPA at 55° C. to 95° C., particularly preferably at about 70° C., until the reaction is complete (2 h to 24 h). The reaction mixture is subsequently diluted with water, with the product generally precipitating cleanly in crystalline form.

Compared with the processes known hitherto, the process according to the invention is significantly simpler to carry out and proceeds with significantly increased yield. Furthermore, further product purification is generally not necessary. It is therefore to be preferred over the known processes both from an economic and ecological point of view.

If the product obtained is an N-arylated cycloimide which contains one or more nitro group(s) in the aryl moiety, the nitro group(s) present can be reduced in a simple manner to (the) amino group(s) (see step 2 of Example 1). In this way, for example, N-(aminophenyl)cycloimide compounds may be present which can then be converted into further valuable compounds.

The invention thus furthermore relates to a process for the preparation of substituted N-(aminoaryl)cycloimide compounds which is characterised in that (a) firstly an aryl compound containing at least one nitro group is reacted with a dicarboxylic acid in the presence of polyphosphoric acid to give the corresponding N-(nitroaryl)cycloimide compound and (b) the resultant N-(nitroaryl)cycloimide compound is subsequently reduced to the corresponding N-(aminoaryl)cycloimide compound. In this way, preferably N-(aminophenyl)cycloimide compounds, particularly preferably N-(4-aminophenyl)cycloimide compounds, are prepared. Suitable reducing agents for the reduction of the nitro group to the amino group are, for example, Raney nickel/hydrogen (RaNi/H₂) and palladium-on-carbon/hydrogen (Pd—C/H₂). Preference is given to the use of Raney nickel/hydrogen. Suitable solvents for carrying out the reduction are, for example, tetrahydrofuran (THF) and/or methanol.

Mention may be made here by way of example of the preparation of 1-(4-nitrophenyl)piperidine-2,6-diones, 1-(4-nitrophenyl)pyrrole-2,5-diones or 1-(4-nitrophenyl)pyrrolidine-2,5-diones and reduction thereof to 1-(4-amino-phenyl)piperidine-2,6-diones, 1-(4-aminophenyl)pyrrole-2,5-diones or 1-(4-aminophenyl)pyrrolidine-2,5-diones respectively. These compounds are valuable intermediates which can be converted further into pharmacologically active compounds, in particular into inhibitors of coagulation factor Xa. At this point, mention may be made by way of example of the conversion of 1-(4-nitrophenyl)piperidine-2,6-dione into (2-(3-carbamimidoylphenoxy)-N-[4-(2,6-dioxopiperidin-1-yl)phenyl]-2-phenylacetamide) which is described in the pending German patent application No. 101 023 22.

The examples, without being restricted thereto, explain the invention.

### EXAMPLE 1

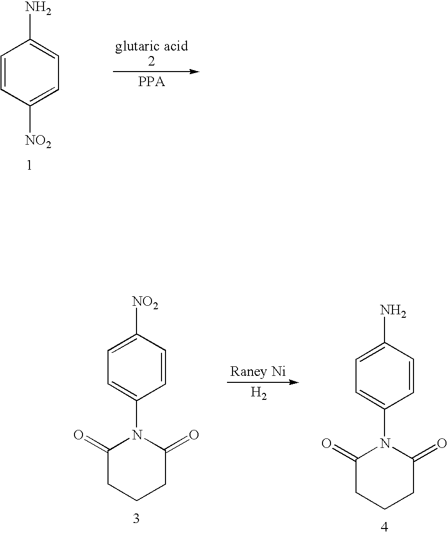

Step 1: 10.0 g (0.072 mol) of 4-nitroaniline 1and 9.512 g (0.072 mol) of glutaric acid 2 are stirred for 12 h at 80° C. in 50.0 g of poly-phosphoric acid. After cooling, 500 mL of water are added with stirring. The resultant precipitate is filtered off with suction, rinsed with water and dried under reduced pressure at 60° C., giving 16.3 g (96.7%) of 1-(4-nitrophenyl)piperidine-2,6-dione 3 having a melting point of 207-209° C.

¹H-NMR (DMSO-d6): 8.30 (d, J=8.8, 2H), 7.46 (d, J=8.8, 2H), 2.79 (t, J=7.9, 4H), 2.03 (m, J=7.9, 2H).

Step 2: 10.0 g (0.043 mol) of 1-(4-nitrophenyl)piperidine-2,6-dione 3 are dissolved in 100 mL of tetrahydrofuran, 1.0 g of RaNi/H₂ is added, and the mixture is hydrogenated using hydrogen at atmospheric pressure with stirring. After uptake of hydrogen has taken place, the catalyst is filtered off, and the resultant reaction-mixture solution is evaporated. The residue is recrystallised from diethyl ether, giving 7.4 g (84.9%) of 1-(4-aminophenyl)piperidine-2,6-dione 4 having a melting point of 214-215° C.

¹H-NMR (DMSO-d6): 6.67 (d, J=8.8, 2H), 6.53 (d, J=8.8, 2H), 5.11 (s-br, 2H), 2.67 (t, J=7.9, 4H), 1.92 (m, J=7.9, 2H).

### EXAMPLE 2

Using the correspondingly substituted aniline and glutaric acid, 3,3-disubstituted glutaric acid, succinic acid or maleic acid, the following compounds are prepared analogously to the process described as step 1 in Example 1:
1-(2-methyl-4-nitrophenyl)piperidine-2,6-dione (1)
1-(2-chloro-4-nitrophenyl)piperidine-2,6-dione (2)
1-(2-methoxy-4-nitrophenyl)piperidine-2,6-dione (3)
1-(2-bromo-4-nitrophenyl)piperidine-2,6-dione (4)
1-(2,4-dinitrophenyl)piperidine-2,6-dione (5)
1-(2-triflouromethyl-4-nitrophenyl)piperidine-2,6-dione (6)
1-(3-triflouromethyl-4-nitrophenyl)piperidine-2,6-dione (7)
1-(2,6-dichloro-4-nitrophenyl)piperidine-2,6-dione (8)
1-(2-phenyl-4-nitrophenyl)piperidine-2,6-dione (9)
4,4-dimethyl-1-(4-nitrophenyl)piperidine-2,6-dione (10)
1-(3-nitrophenyl)piperidine-2,6-dione (11)
1-(2-nitrophenyl)piperidine-2,6-dione (12)
1-(4-ethylphenyl)piperidine-2,6-dione (13)
1-(3-chlorophenyl)piperidine-2,6-dione (14)
1-(4-chlorophenyl)piperidine-2,6-dione (15)
1-(4-nitrophenyl)pyrrolidine-2,5-dione (16)
1-(2-chloro-4-nitrophenyl)pyrrolidine-2,5-dione (17)
1-(2,4-dinitrophenyl)pyrrolidine-2,5-dione (18)
1-(2-methyl-4-nitrophenyl)pyrrolidine-2,5-dione (19)
1-(2,6-dichloro-4-nitrophenyl)pyrrolidine-2,5-dione (20)
1-(2-bromo-4-nitrophenyl)pyrrolidine-2,5-dione (21)
1-(2-benzoyl-4-nitrophenyl)pyrrolidine-2,5-dione (22)
1-(2-methoxy-4-nitrophenyl)pyrrolidine-2,5-dione (23)
1-(2-carboxy-4-nitrophenyl)pyrrolidine-2,5-dione (24)
1-(2-triflouromethyl-4-nitrophenyl)pyrrolidine-2,5-dione (25)
1-(3-triflouromethyl-4-nitrophenyl)pyrrolidine-2,5-dione (26)
1-(2-phenyl-4-nitrophenyl)pyrrolidine-2,5-dione (27)
1-(4-nitrophenyl)pyrrole-2,5-dione (28)
1-(2-triflouromethyl-4-nitrophenyl )pyrrole-2,5-dione (29)

### EXAMPLE 3

A selection of the compounds prepared in accordance with Example 2 are converted into the compounds mentioned below analogously to the processes described as step 2 in Example 1:
compound 6 into 1-(2-triflouromethyl-4-aminophenyl)piperidine-2,6-dione (30)
compound 3 into 1-(2-methoxy-4-aminophenyl)piperidine-2,6-dione (31)
compound 1 into 1-(2-methyl-4-aminophenyl)piperidine-2,6-dione (32)
compound 7 into 1-(3-triflouromethyl-4-aminophenyl)piperidine-2,6-dione (33)
compound 10 into 4,4-dimethyl-1-(4-aminophenyl)piperidine-2,6-dione (34)
compound 16 into 1-(4-aminophenyl)pyrrolidine-2,5-dione (35)
compound 17 into 1-(2-chloro-4-aminophenyl)pyrrolidine-2,5-dione (36)
compound 18 into 1-(2,4-diaminophenyl)pyrrolidine-2,5-dione (37)
compound 19 into 1-(2-methyl-4-aminophenyl)pyrrolidine-2,5-dione (38)
compound 20 into 1-(2,6-dichloro-4-aminophenyl)pyrrolidine-2,5-dione (39)
compound 23 into 1-(2-methoxy-4-aminophenyl)pyrrolidine-2,5-dione (40)
compound 26 into 1-(3-triflouromethyl-4-aminophenyl)pyrrolidine-2,5-dione (41)
compound 25 into 1-(2-triflouromethyl-4-aminophenyl)pyrrolidine-2,5-dione (42)

All compounds prepared were characterised by mass spectroscopy. Furthermore, the solid point (SP) of all compounds was determined. The results are shown in Table 1.

Mass spectrometry (MS): EI (electron impact ionisation) M⁺
FAB (fast atom bombardment) (M+H)⁺

Above and below, all temperatures are indicated in ° C.

TABLE 1

| | | | | MS |
|---|---|---|---|---|
| | | MW | SP | EI/ |
| No. | chemical structure | [g/mol]; | [° C.] | FAB] |
| 1 | 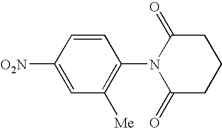 | 248.24 | 175-179 | 249 |
| 2 | 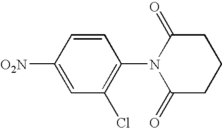 | 268.66 | 179-182 | 269 |
| 3 | 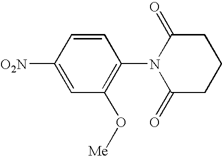 | 264.24 | 172-177 | 265 |
| 4 | 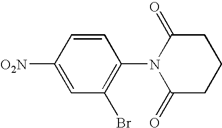 | 313.11 | 117-120 | 313 |
| 5 | 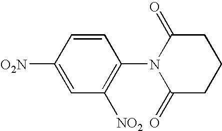 | 279.21 | 169-170 | 280 |
| 6 | 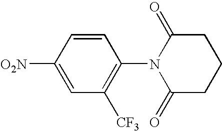 | 302.21 | 176-177 | 302 |
| 7 | 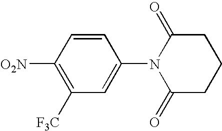 | 302.21 | 125-126 | 302 |
| 8 | 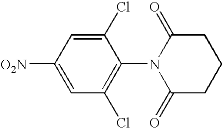 | 303.10 | 206-207 | 304 |
| 9 | 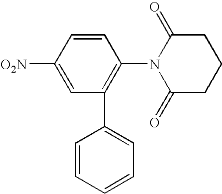 | 310.31 | 139-140 | 310 |
| 10 | 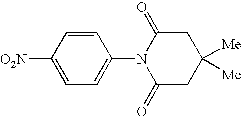 | 262.27 | 201-202 | 262 |
| 11 | 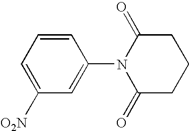 | 234.21 | 205-206 | 235 |
| 12 | 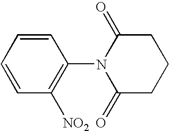 | 234.21 | 97-98 | 235 |
| 13 | 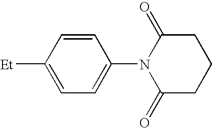 | 217.27 | 135-136 | 218 |
| 14 | 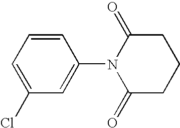 | 223.66 | 128-129 | 224 |
| 15 | 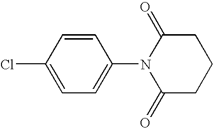 | 223.66 | 143-144 | 224 |
| 16 | 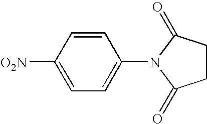 | 220.19 | 215-217 | 220 |
| 17 | 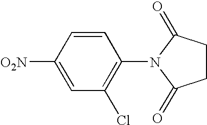 | 254.63 | 160-162 | 254 |
| 18 | 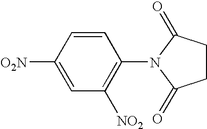 | 265.18 | 220-222 | 265 |
| 19 | 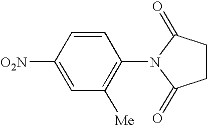 | 234.21 | 205-207 | 234 |
| 20 | 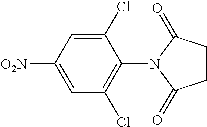 | 289.08 | 199-201 | 288 |
| 21 | 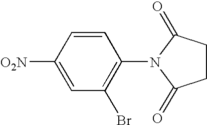 | 299.08 | 169-171 | 298 |
| 22 | 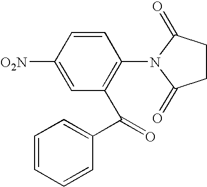 | 324.30 | 174-176 | 324 |
| 23 | 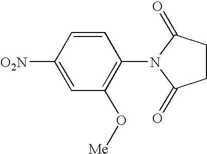 | 250.21 | 167-169 | 250 |
| 24 | 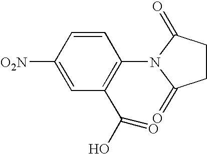 | 264.20 | 246-250 | 264 |
| 25 | 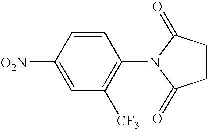 | 288.19 | 205-207 | 288 |
| 26 | 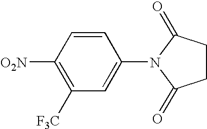 | 288.19 | 106-107 | 288 |
| 27 | 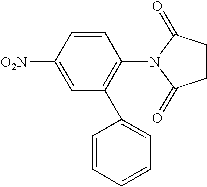 | 296.29 | 135-137 | 296 |
| 28 | 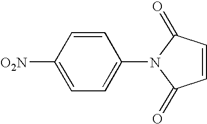 | 218.17 | 170-171 | 218 |
| 29 | 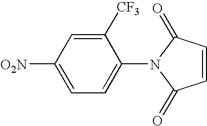 | 286.17 | 109-111 | 287 |
| 30 | 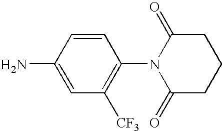 | 272.23 | 201-202 | 273 |
| 31 | 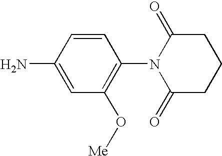 | 234.26 | 120-121 | 234 |
| 32 | 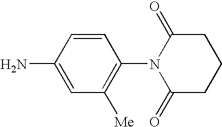 | 218.26 | 153-154 | 218 |
| 33 | 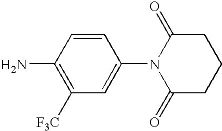 | 272.23 | 169-170 | 273 |
| 34 | 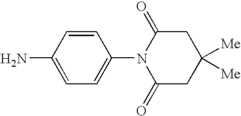 | 232.29 | 185-186 | 233 |
| 35 | 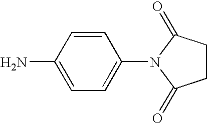 | 190.20 | 240-242 | 190 |
| 36 | 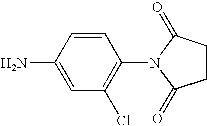 | 224.65 | 230-232 | 224 |
| 37 | 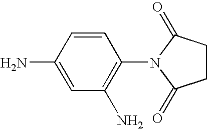 | 205.22 | 240-242 | 205 |
| 38 | 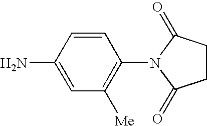 | 204.23 | 174-175 | 204 |
| 39 | 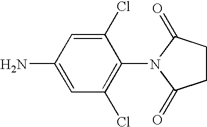 | 259.09 | 255-257 | 258 |
| 40 | 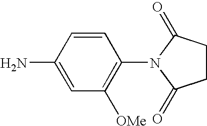 | 220.23 | 161-163 | 220 |
| 41 | 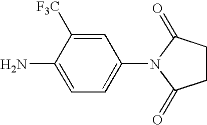 | 258.20 | 115-117 | 258 |
| 42 | 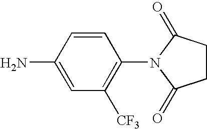 | 258.20 | 157-159 | 258 |

1. Process for the preparation of N-substituted cyclic imides, which is characterised in that a primary amine is reacted with a dicarboxylic acid in the presence of polyphosphoric acid

2. Process according to claim 1, characterised in that the primary amine employed is substituted or unsubstituted aniline

3. Process according to claim 2, characterised in that the primary amine employed is a compound of the general formula I 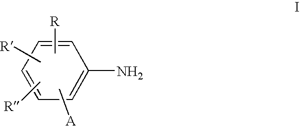
in which
R, R′, R″, independently of one another, are H, F, Cl, Br, I, alkyl, O-alkyl, —(C═O)alkyl, O—(C═O)alkyl, aryl, COOH, —(C═O)aryl, OCF₃, CF₃, CN, OCHF₂ or2,3-CH═CH—CH═CH—,
A is H, NO₂, NH₂ or NH—(C═O)—R¹,
alkyl is unbranched or branched alkyl having 1-6 C atoms,
aryl is phenyl or thienyl, each of which is unsubstituted or monosubstituted by alkyl, O-alkyl, CF₃,
R¹ is 2-phenoxy-2-aryl(or alkyl)acetamide or 2-phenylamino-2-aryl(or alkyl)acetamide

4. Process according to claim 1, characterised in that the dicarboxylic acid employed is maleic acid, succinic acid or substituted or unsubstituted glutaric acid

5. Process according to claim 1, characterised in that equimolar amounts of primary amine and dicarboxylic acid are reacted with one another

6. Process for the preparation of substituted N-(aminoaryl)cycloimide compounds, which is characterised in that
(a) firstly an aryl compound containing at least one nitro group is reacted with a dicarboxylic acid in the presence of polyphosphoric acid to give the corresponding N-(nitroaryl)cycloimide compound and
(b) the resultant N-(nitroaryl)cycloimide compound is subsequently reduced to the corresponding N-(aminoaryl)cycloimide compound

7. Process according to claim 6, is characterised in that the N-(nitroaryl)cycloimide compound reacted in step (a) is an N-(nitrophenyl)cycloimide compound

8. Process according to claim 6, characterised in that the reduction of the nitro group in (b) is carried out using Raney nickel/hydrogen
